(19) United States
(12) Patent Application Publication
(10) Pub. No.: US 2019/0297952 A1
(43) Pub. Date: Oct. 3, 2019

(54) CARTRIDGE, ATOMIZING ASSEMBLY AND ELECTRONIC CIGARETTE THEREOF

(71) Applicant: Changzhou Patent Electronic Technology Co., LTD, Changzhou (CN)

(72) Inventor: Weihua QIU, Changzhou (CN)

(21) Appl. No.: 16/447,869

(22) Filed: Jun. 20, 2019

Related U.S. Application Data
(63) Continuation-in-part of application No. PCT/CN2017/115022, filed on Dec. 7, 2017.

(30) Foreign Application Priority Data
Dec. 20, 2016 (CN) 201621406878.6
Dec. 20, 2016 (CN) 201621406879.0
Dec. 20, 2016 (CN) 201621411023.2

(51) Int. Cl. A24F 47/00; B05B 7/16; A61M 11/04

(52) U.S. Cl. CPC A24F 47/008; A61M 11/042; B05B 7/1686

(57) ABSTRACT

A cartridge, an atomizing assembly and an electronic cigarette thereof are provided. The cartridge includes a cartridge housing, a reservoir chamber, an intake passage, and a smoke outlet passage; the reservoir chamber, the intake passage, and the smoke outlet passage are received in the cartridge housing, the intake passage and the outlet passage are disposed at the same side of the reservoir chamber. And the same side placement of the intake passage and the smoke outlet passage is compact in structure, the air inlet passage and the smoke outlet passage are arranged next to each other, the length of the air passage from the air inlet to the air outlet is shortened. Therefore, the problem that the user needs to pump vigorously to suck out the smoke is effectively solved, and the user's inhale is more convenient and labor-saving.

### CROSS-REFERENCE TO RELATED APPLICATIONS

The present application is a continuation in part of international application No. PCT/CN2017/115022 filed on Dec. 7, 2017, and claims priority to Chinese patent application Nos. 201621406878.6, 201621406879.0, and 201621411023.2 filed on Dec. 20, 2016, and the entire disclosures of the foregoing applications are incorporated herein by reference.

### TECHNOLOGY FIELD

The present invention relates to an electronic cigarette, in particular to a cartridge, an atomizing assembly and electronic cigarette thereof.

### BACKGROUND

Conventional electronic cigarette generally includes a cartridge, an atomizer electronic and an electronic control assembly. Atomizer typically includes a heating element, a guiding member, an intake passage and the exhaust passage. The setting of the intake and exhaust passages of the electronic cigarette will affect the final smoke effect, the existing electronic cigarette inlet and outlet passages are not ideally set, which affects the user's inhale.

### SUMMARY

In order to solve the above problem of the arrangement of the intake passage and the smoke outlet passage, the technical solution adopted by the present disclosure is:

The present disclosure provided with a cartridge, the cartridge includes a cartridge housing, a reservoir chamber, an intake passage, and a smoke outlet passage; the reservoir chamber, the intake passage, and the smoke outlet passage are received in the cartridge housing, the intake passage and the outlet passage are disposed at the same side of the reservoir chamber.

In one embodiment, the reservoir chamber, the intake passage and the smoke outlet passage are sequentially arranged side by side.

In one embodiment, the cartridge includes a top wall and a peripheral wall, the top wall and a peripheral wall are correspondingly enclosing a reservoir chamber, the peripheral wall of the cartridge is provided with a smoke outlet passage and an intake passage the same side.

In one embodiment, an air inlet is defined at the peripheral wall of the cartridge, the air inlet is in communication with the Intake passage.

In one embodiment, a partition wall is formed between the intake passage and the smoke outlet passage.

In one embodiment, the lower end passage of the smoke outlet passage is narrowed relative to the upper end passage.

In one embodiment, an inserting member is disposed in the smoke outlet passage, after the inserting member is inserted into the smoke outlet passage, the smoke outlet passage is substantially generally trumpet-shaped having a bigger, upper part and a smaller, lower part.

In one embodiment, the cartridge further comprising a main body, an upper cover and a bottom cover, the upper cover is provided with a smoke outlet, the smoke outlet passage is in communication with the smoke outlet.

In one embodiment, the inner wall of the outlet passage is provided with a slope or a bump, the outlet passage is provided with a slope or a projection, so that the outlet passage is substantially generally trumpet-shaped having a bigger, upper part and a smaller, lower part.

In one embodiment, the two inserting members have a cylindrical shape at the lower end and a wedge shape at the upper end, or the insert is a cylinder, the outer wall of the insert is axially attached to the inner wall of the outlet passage, the inserting member has a tapered hole penetrating vertically inside, the diameter of the tapered hole gradually decreases from top to bottom.

Further, the present disclosure provided with an atomizing assembly. The atomizing assembly includes any one of the above cartridges, and further includes an atomizer detachably secured to the bottom end of the cartridge.

In one embodiment, the atomizing head includes an atomizing head housing, a heating element, and a liquid guiding member, the atomizing head housing includes a housing body and an atomizing head upper cover, a cavity is defined inside the housing body, the heating element and the liquid guiding member are both located in the cavity, the liquid guiding member is sleeved on the outer circumference of the heating element, the atomizing head upper cover is provided with a liquid inlet hole in communication with the cavity, the liquid guiding member is located at the lower end of the liquid inlet hole to ensure that the smoke liquid flowing through the liquid inlet hole can be introduced into the liquid guiding member, a blocking portion is disposed between the heating element and the liquid inlet hole, the upper end surface of the blocking portion abuts against the lower end surface of the atomizing head upper cover, the lower end surface of the blocking portion abuts against the upper end surface of the liquid guiding member to prevent the smoke liquid flowing through the liquid inlet hole from directly contacting the heating element without the liquid guiding member.

In one embodiment, the atomizer includes an atomizing head main body, the atomizing head main body is provided with an atomizing head mounting hole, the atomizing head is disposed in the atomizing head mounting hole.

In one embodiment, the outer peripheral wall of the housing body defines a limiting portion.

In one embodiment, the atomizing head mounting hole is provided with a matching portion that is fixedly coupled with the limiting portion, the atomizing head is fixed in the atomizing head mounting hole by the matching portion.

In one embodiment, the limiting portion is an L-shaped groove formed on the outer peripheral wall of the housing body; corresponding to the position of the L-shaped groove, the inner peripheral wall of the atomizing head mounting hole is provided with a protrusion that cooperates with the L-shaped groove, the protrusion is the matching portion; or, the limiting portion is a protrusion formed on the outer peripheral wall of the housing body; corresponding to the position of the protrusion, the inner peripheral wall of the atomizing head mounting hole is provided with an L-shaped groove that cooperates with the protrusion, the L-shaped groove is the fitting portion.

Further, the present disclosure provided with an atomizing assembly, the atomizing assembly includes any one of the above cartridges, and further includes an atomizer provided with an atomizing heating channel, one end of the atomization heating passage is in communication with the intake passage, the other end is in communication with the smoke outlet passage.

In one embodiment, the atomizing head includes a reservoir tube and an inner sleeve sleeved in the liquid storage tube, the inner sleeve includes an atomizing tube, the pipe of the atomizing tube is an atomizing heating channel.

In one embodiment, the atomizing head includes a heating element and a liquid guiding element, the heating element is wrapped around the outer circumference of the liquid guiding member, or the liquid guiding member is wrapped around the outer circumference of the heating element.

Further, the present disclosure provided with an electronic cigarette including any one of the above atomizing assemblies.

The beneficial effects of the present disclosure are:

The same side of the cartridge forms a smoke outlet passage and an intake passage, both the intake passage and the smoke outlet passage are placed in the cartridge, thereby simplifying the originally crowded atomizer structure. And the same side placement of the intake passage and the smoke outlet passage is compact in structure, the air inlet passage and the smoke outlet passage are arranged next to each other, the length of the air passage from the air inlet to the air outlet is shortened. Therefore, the problem that the user needs to pump vigorously to suck out the smoke is effectively solved, and the user's inhale is more convenient and labor-saving. In one embodiment, the lower end passage of the outlet passage is narrowed relative to the upper end passage thereof, so that a negative pressure is more easily formed in the smoke outlet passage, the suction effect is better.

### BRIEF DESCRIPTION OF THE DRAWINGS

The accompanying drawings are included to provide a further understanding of the disclosure and are used in explaining the disclosure together with the specific embodiments below. However, it should not be construed as limiting the invention. In the drawing,

FIG. 1 is a cross-sectional view of an electronic cigarette of a first embodiment;

FIG. 2 is an exploded perspective view of one embodiment of the cartridge of the first embodiment.

FIG. 3 is a cross-sectional view along line A-A of one embodiment of the cartridge of the first embodiment.

FIG. 4 is a cross-sectional view taken along line B-B of one embodiment of the cartridge of the first embodiment;

FIG. 5 is a cross-sectional view showing another embodiment of the cartridge of the first embodiment;

FIG. 6 is a cross-sectional view of the electronic cigarette of a second embodiment;

FIG. 7 is a cross-sectional view of another perspective of the electronic cigarette shown in FIG. 6;

FIG. 8 is a perspective view of the atomizing head in the electronic cigarette shown in FIG. 6;

FIG. 9 is an exploded view of the atomizing head shown in FIG. 8;

FIG. 10 is a plan view of the atomizing head shown in FIG. 8;

FIG. 11 is a cross-sectional view of the atomizing head shown in FIG. 8;

FIG. 12 is a cross-sectional view of another perspective view of the atomizing head shown in FIG. 8.

FIG. 13 is a cross-sectional view of the atomizing head in a third embodiment.

FIG. 14 is a cross-sectional view showing a part of the structure of the atomizing head in the third embodiment.

FIG. 15 is an exploded view of the atomizing head in the third embodiment.

FIG. 16 is a cross-sectional view of the cartridge of the third embodiment;

FIG. 17 is an exploded view of the cartridge in the third embodiment.

FIG. 18 is a cross-sectional view of the electronic cigarette in the third embodiment.

The following table list various components and reference numerals thereof.

| | |
|---|---|
| Main body 10 | Bottom cover 12 |
| upper cover 11 | Buckle 30 |
| Mounting hole 107 | Decorative strip 108 |
| Smoke outlet 110 | Partition wall 106 |
| Slope 14 | Sensor 6, 46b |
| Electrical connecting member 4 | Rubber stopper 5 |
| Connecting portion 60 | Electronic control assembly 7 |
| Outer housing 8 | USB connection hole 80 |
| USB interface 9 | Smoke guiding cover 25a |
| Electronic control assembly 40a | Baffle 26a |
| Button hole 11a | Atomizing component 30a |
| USB port 12a | Mounting bracket 41a |
| Trigger button 13a | Circuit board: 42a |
| Top wall 21a | Power source 45a |
| peripheral wall 22a | Insulating member 313a |
| receiving groove 211a | Atomizing head housing 11b |
| Atomizing upper head cover 19b | housing body 20b |
| reservoir chamber hole 25b | Atomizing head main body 31b |
| Battery assembly 30b | shell 40b |
| Sealing member 23b | Air flow passage 44b |
| cartridge housing 211b | |
| Mounting groove: 105 | Intake passage 102, 221a |
| Locking hole 109 | Smoke outlet passage 103, 222a, 43b |
| Inserting member 13 | Atomizing head 2, 31a, 10b |
| Main board 70 | Liquid guiding member 12b |
| Battery 71 | Heating element 315a, 13b |
| Main body: 3 | Reservoir chamber 101, 23a, 24b |
| Recess 50 | Air inlet 104, 24a, 42b |
| Shell 10a | Conductor: 314a |
| Liquid storage gap 316a | Atomizing tube 3121a |
| Smoke guiding hole 251a | Liquid guiding tube 3122a |
| Liquid inlet 261a | Smoke outlet 3123a |
| Main body 301a | Liquid injecting port: 3124a |
| reservoir tube 311a | Liquid inlet hole 3125a |
| Inner sleeve 312a | Liquid pipe 3126a |
| Liquid inlet hole 16b | Limiting portion 111 |
| Blocking portion 17b | Atomizing head mounting hole 311b |
| Smoking outlet 41b | Air outlet 18b |
| Circuit board 45b | Atomizing head Air passage 131b |
| Matching portion 113 | Sealing gasket: 47b |

### DETAILED DESCRIPTION OF EMBODIMENTS

The specific embodiments of the present disclosure will be described in detail below with reference to the accompanying drawings. It is to be understood that the specific embodiments described herein are merely illustrative and not restrictive.

### The First Embodiment

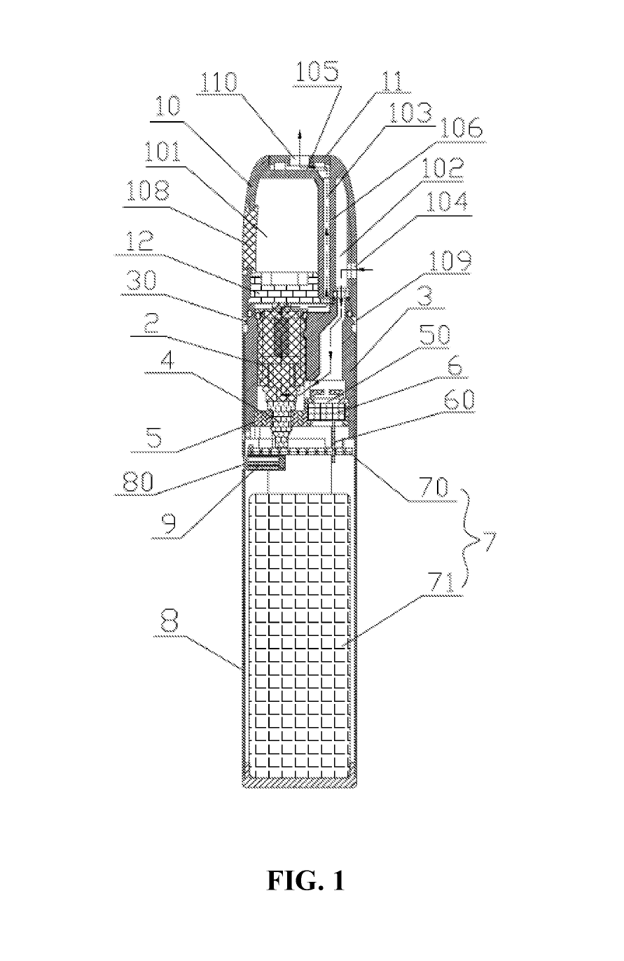

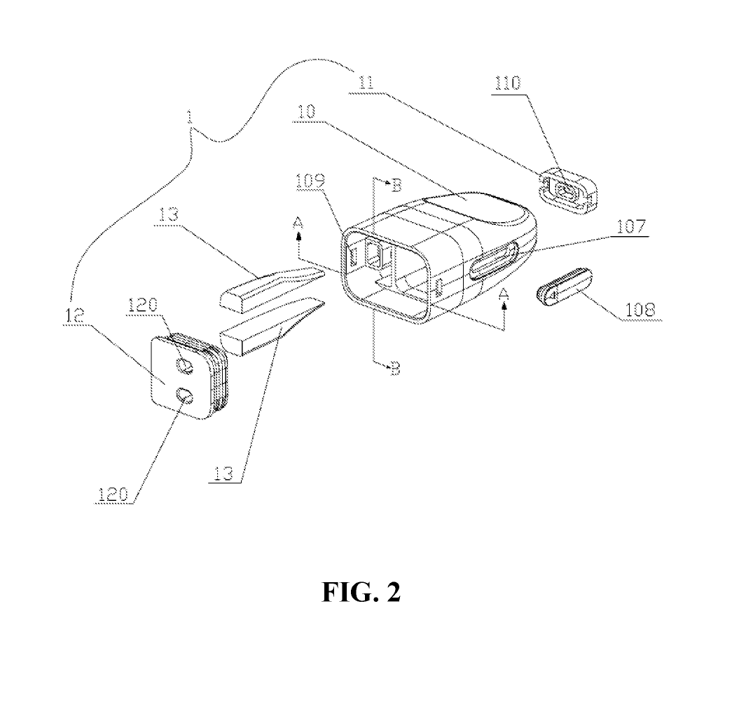

Referring to FIGS. 1 and 2, the electronic cigarette of the present embodiment includes: a outer housing 8, a cartridge 1 mounted on top of the outer housing 8, an atomizing assembly (not labeled) mounted within the outer housing 8, and an electric control assembly 7. The cartridge 1 provides smoke liquid to the atomizing assembly, the atomizing assembly atomizes the smoke liquid flowed therein, the electric control assembly 7 is mounted at the lower end of the atomizing assembly, and is electrically connected to the atomizing assembly to supply power to the atomizing assembly. A USB connection hole 80 is defined in the peripheral wall of the outer housing 8 for charging the electronic control assembly 7 by an external power source.

The cartridge 1 includes a main body 10, upper cover 11 and a bottom cover 12. The main body 10, the upper cover 11 and the bottom cover 12 constitute a cartridge housing (not shown), the top of the main body 10 defines a mounting groove 105, the upper cover 11 is mounted in the mounting groove 105, and a smoke outlet 110 is defined at the intermediate position of the upper cover 11. The main body 10 is provided with a reservoir chamber 101 therein. The reservoir chamber 101 is a top closed, bottom open structure, the bottom cover 12 encloses the bottom opening of the reservoir chamber 101. In addition, an intake passage 102 and a smoke outlet passage 103 are formed in the axial direction inside the main body 10. The intake passage 102 and the smoke outlet passage 103 are disposed on the same side of the reservoir chamber 101. Further, the reservoir chamber 101, the intake passage 102 and the smoke outlet passage 103 are sequentially arranged side by side. The diameter of the intake passage 102 is the same as above and below, the outlet of the smoke outlet passage 103 is the same as above and below. Optionally, the inlet passage 102 and the smoke outlet passage 103 have a circular or square cross-section; the side wall of the main body 10 is provided with an air inlet 104 in communication with the intake passage 102, the intake passage 102 and the smoke outlet passage 103 are both located on the same side of the cartridge 1, the smoke outlet passage 103 is in communication with the smoke outlet 110. By the above arrangement, the length of the air path from the air inlet 104 to the smoke outlet 110 is shortened, thereby effectively solving the problem that the user needs to pump vigorously to suck out the smoke, and the user is more convenient and labor-saving. In addition, the side wall of the reservoir chamber 101 is provided with a mounting hole 107, a decorative strip 108 is mounted in the mounting hole 107, and the decorative strip 108 is made of transparent material. Therefore, the position of the e-cigarette liquid in the reservoir chamber 101 can be observed through the decorative strip 108, and the amount of the liquid in the reservoir chamber 101 can be easily checked.

Specifically, a partition wall 106 is formed between the intake passage 102 and the smoke outlet passage 103. The intake passage 102 is adjacent to the outer peripheral surface of the main body 10, the smoke outlet passage 103 is adjacent to the reservoir chamber 101. The intake passage 102 extends axially from the bottom of the peripheral wall of the main body 10 by a predetermined height, the peripheral wall of the main body 10 is provided with an air inlet 104 corresponding to and in communication with the intake passage 102. In this embodiment, the air inlet 104 is in communication with the top end of the intake passage, such that after being supplied from the air inlet 104, air can flow out from the bottom of the peripheral wall along the intake passage 102. The smoke outlet passage 103 extends axially from the bottom of the peripheral wall to the top of the peripheral wall, and is in communication with the mounting groove 105, so that the airflow flows from the bottom of the smoke outlet passage 103 and flows out from the top of the peripheral wall to the mounting groove 105, the user then inhales through the smoke outlet 110. Thus, this embodiment realizes air enters and smoke flow out on the same side of the cartridge 1, the airflow enters at the upper end and flows out at the upper end, thereby the disclosure solves the problem that the air inlet hole of the existing electronic cigarette is opened at the bottom of the outer casing to cause the e-cigarette liquide-cigarette liquid to leak easily when the air enters in. In one embodiment, the intake passage 102 and the smoke outlet passage 103 are both formed by the inner cavity of the tubular body, the intake passage 102 and the smoke outlet passage 103 are arranged side by side. In another embodiments, the intake passage 102 is an inner chamber of the tubular body, the smoke outlet passage 103 is disposed around the intake passage 102. Further, the intake passage 102 is housed in the main body 10 and a part of the passage wall of the intake passage 102 is connected to the inner wall of the main body 10, and the other part of the passage wall is surrounded by the smoke outlet passage 103. Specifically, the space between the passage wall of the intake passage 102, the inner wall of the main body 10 and the wall of the reservoir chamber 101 constitutes a smoke outlet passage 103.

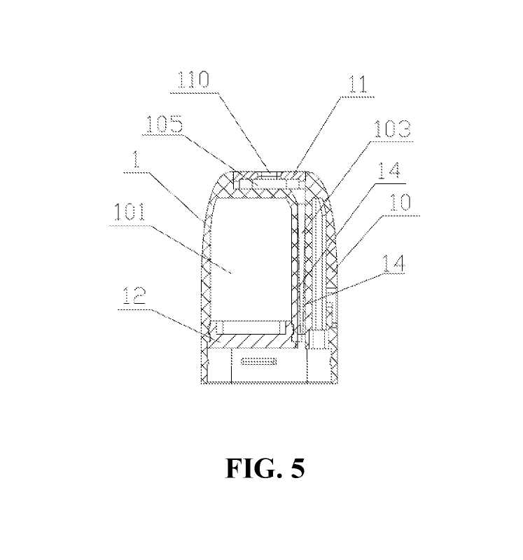

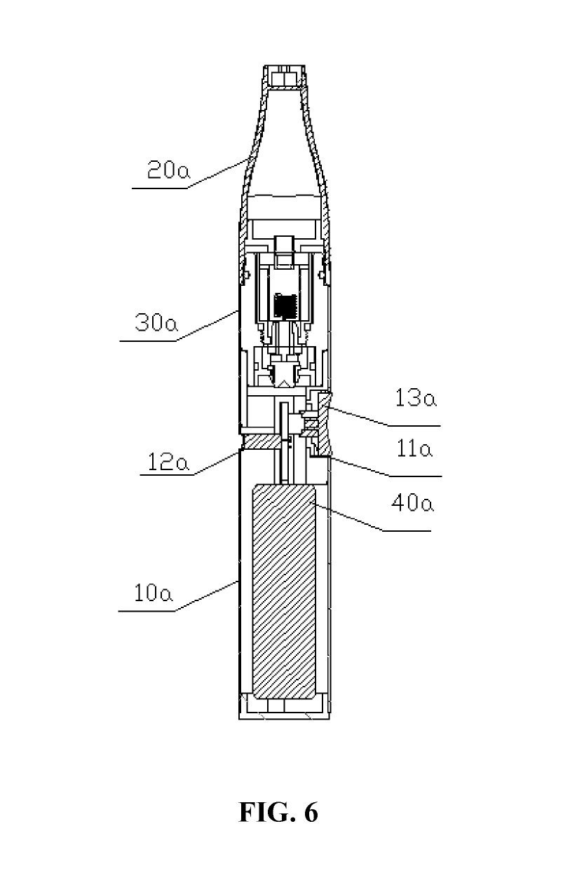

Referring to FIG. 1, FIG. 5 and FIG. 6, the atomizing assembly is mounted at the bottom of the cartridge 1, the bottom of the atomizing assembly is connected to the outlet of the inlet passage 102, and the top of the atomizing assembly is connected to the inlet of the smoke outlet passage 103. The atomizing assembly atomizes the smoke liquid flowing from the cartridge 1 into the reservoir 101 to generate smoke, so that the smoke can be carried out through the airflow. The atomizing assembly includes a main body 3 and an atomizing head 2 installed in the main body 3, the main body 3 has a tubular shape and is installed in the outer casing 8. The top of the main body 3 is detachably assembled to the bottom of the cartridge 1. Specifically, the bottom surface of the main body 10 is provided with a locking hole 109, both sides of the top of the main body 3 are protruded to form a buckle 30 respectively. Each of the buckles 30 is correspondingly inserted into one of the locking holes 109.

The provision of the buckle 30 on the main body 3 enables the size of the buckle 30 to be made larger, thereby effectively preventing the cartridge 1 from being detached from the atomizing assembly. The atomizing head 2 is provided with an atomizing heating channel (not shown). One end of the atomizing heating passage communicates with the intake passage 102, and the other end communicates with the smoke outlet passage 103. Specifically, an air passage gap (not shown) is disposed between the intake passage 102 and the atomizing head 2, and between the air outlet passage 103 and the atomizing heating passage, respectively. Thereby, the intake passage 102 is in communication with the smoke outlet passage 103.

The structure of the atomizing head 2 in this embodiment is the same as that of the atomizing head in the third embodiment. The detailed structure is described in the third embodiment, details are not described herein again. It can be understood that in other embodiments, the atomizing head 2 can adopt other structures, and is not limited thereto.

Further, the bottom of the main body 3 is provided with a rubber plug 5, the rubber plug 5 is provided with a perforation corresponding to the positive electrode contact 23 (not shown). An electrical connecting member 4 is arranged in the perforation. The upper end of the electrical connecting member 4 is connected to the positive contact 23, the lower end of the electrical connecting member 4 protrudes from the bottom of the rubber plug 5. In addition, the rubber plug 5 is provided with a recess 50 corresponding to the intake passage 102, the recess 50 is in communication with the intake passage 102. A sensor 6 is mounted within the recess 50, the sensor 6 can be selected as an air pressure sensor or an air flow sensor. The sensor 6 projects downwardly to form a connecting portion 60 which extends beyond the bottom of the rubber plug 5.

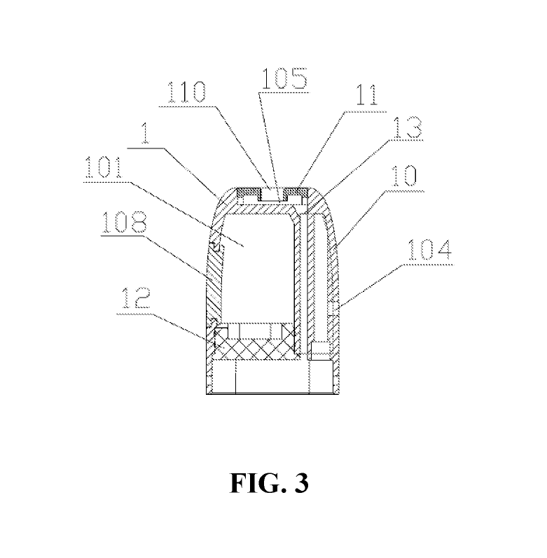

Please referring to FIGS. 2-4, the present embodiment changes the internal structure of the smoke outlet passage 103 by narrowing the lower end passage of the smoke outlet passage 103 with respect to the upper end passage thereof, thereby optimizing the suction effect and improving the sensitivity of the sensor 6.

In one of the embodiments, an external member such as the Inserting member 13 is inserted into the smoke outlet passage 103 to narrow the lower end passage of the smoke outlet passage 103 with respect to the upper end passage thereof.

In another embodiment, a slope or a bump is provided in the passage wall of the smoke outlet passage 103, and the lower end passage of the smoke outlet passage 103 is narrowed relative to the upper end passage thereof by directly changing the shape of the passage wall.

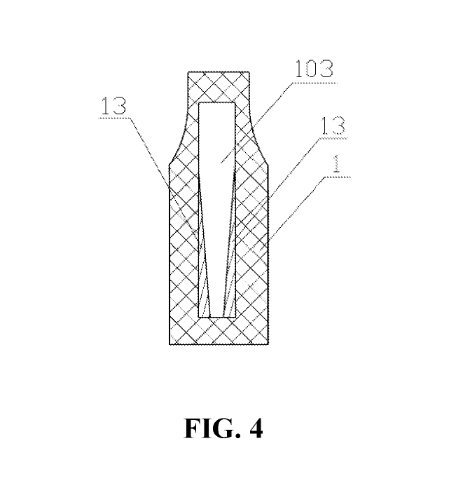

The following is a detailed description. First, please refer to FIG. 2 and FIG. 4. In this embodiment, two oppositely disposed Inserting members 13 are disposed in the smoke outlet passage 103, the lower end passage of the smoke outlet passage 103 is narrowed relative to the upper end passage thereof, thereby making it easier to form negative pressure in the smoke outlet passage 103, the sensitivity of the sensor 6 is increased after the insertion of the Inserting member 13. The two Inserting members 13 can be selected as a silicone plug, and the opposite sides are disposed on the inner wall of the smoke outlet passage 103 along the axial direction of the smoke outlet passage 103. The Inserting member 13 is interference fit with the smoke outlet passage 103 to fix the Inserting member 13 in the smoke outlet passage 103; or two Inserting members 13 are disposed adjacent to the inner wall of the smoke outlet passage 103. It can be understood that when the cross-section of the smoke outlet passage 103 is square, the Inserting member 13 can be set in different directions. For example, the two Inserting members 13 of FIG. 4 are disposed in the front-rear direction of the smoke outlet passage 103. In another embodiment, the Inserting member 13 is disposed in the left-right direction of the smoke outlet passage 103 (the front-rear direction in the A-A direction in FIG. 2, and the left-right direction in the B-B direction in FIG. 2). Alternatively, two Inserting members 13 are disposed adjacent to the two inner walls of the smoke outlet passage 103. It should be noted that the lengths of the two Inserting members 13 can be the same as the length of the smoke outlet passage 103, or can be shorter than the smoke outlet passage 103. After the two inserting members 13 are added, the smoke outlet passage 103 is substantially generally trumpet-shaped having a bigger, upper part and a smaller, lower part. It can be understood that the two inserting members 13 can also be rubber plugs, TPUs and the like having certain deformation characteristics.

In this embodiment, the two inserting members 13 have a cylindrical shape at the lower end and a wedge shape at the upper end. Thus, when the two Inserting members 13 are inserted into the smoke outlet passage 103, the Inserting member 13 having a wedge shape partially fits the inner wall of the upper end passage of the smoke outlet passage 103. The Inserting member 13 having a cylindrical shape partially fits the inner wall of the lower end of the smoke outlet passage 103, so that a large and small trumpet-shaped air passage structure can be formed, which is favorable for forming a negative pressure during smoking, and the smoking effect is better.

In one embodiment, the Inserting member 13 is one, the outer wall of the Inserting member 13 is axially attached to the inner wall of the smoke outlet passage 103. The inserting member 13 can be selected as a cylinder, the inserting member 13 has a tapered hole extending through the top. The diameter of the tapered hole gradually decreases from top to bottom.

In one of the embodiments, the Inserting members 13 are three, each Inserting member 13 is disposed along the axial direction of the smoke outlet passage 103, the three Inserting members 13 are arranged circumferentially along the inner wall of the smoke outlet passage 103, the Inserting member 13 is generally wedge-shaped with a smaller upper portion and a larger lower portion. The Inserting member 13 is disposed in the smoke outlet passage 103 such that the smoke outlet passage 103 has a large and small trumpet shape. In other embodiments, the Inserting members 13 are four or more, and the arrangement is the same as when the Inserting members 13 are three.

Referring to FIG. 5, in another embodiment, the smoke outlet passage 103 can be directly formed into a structure that is large and small, such that the lower end passage of the smoke outlet passage 103 is narrowed relative to the upper end passage. This structure has the same function as the provision of the Inserting member 13 in the smoke outlet passage 103. Illustratively, by providing two slopes 14 axially opposite each other on the inner wall of the smoke outlet passage 103, optionally, the slope 14 has a wedge shape, so that the smoke outlet passage 103 is generally trumpet-shaped with a larger, upper portion and smaller, lower portion, which is favorable for forming a negative pressure when sucking, the smoking effect is better. In addition, the slope 14 can also be a wedge-shaped bump, and is not limited thereto.

The electronic control assembly 7 includes a main board 70 and a battery 71, the main board 70 is disposed at a lower end of the rubber plug 5. The battery 71 is located at the lower end of the main board 70 and is electrically connected to the main board 70. The lower end of the electrical connecting member 4 abuts against the upper surface of the main board 70 to make electrical contact with the main board 70. Thereby, the electrical connection between the atomizing head 2 and the main board 70 is realized. The connecting portion 60 is inserted into the hole of the main board 70, such that the sensor 6 is connected to the main board 70 to control the operation of the battery 71. When the user smokes, the sensor 6 senses that the negative pressure will start to work, causing the battery 71 to output a voltage to the main board 70, so that the atomizing head 2 operates normally. A USB interface 9 corresponding to the USB connection hole 80 is mounted on the lower surface of the main board 70 for external power supply to charge the battery 71. The rubber plug 5 can create a negative pressure sensing space for the sensor 6 inside the main body 3, and can also prevent the condensed smoke droplets from falling onto the main board 70 and damaging the main board 70. In the embodiment, the rubber plug 5 is a silicone plug. In other embodiments, the rubber plug 5 may also be a rubber plug, and is not limited thereto.

In addition, the components of the cartridge 1, the main body 3 and the atomizing head 2 are closely matched by an O-shaped silicone ring (not shown), thereby effectively preventing the leakage of the electronic cigarette. The atomizing assembly is detached from the cartridge 1 to facilitate replacement of the atomizing head 2.

When smoking, the airflow enters from the air inlet 104, flows into the interior of the main body 3, causes a negative pressure in the main body 3, and then the sensor 6 senses a negative pressure, causing the main board 70 and the atomizing head 2 to operate; meanwhile, the airflow enters the inside of the atomizing head 2 via The bottom of the atomizing head 2, the smoke is taken out from the top of the atomizing head 2 to the smoke outlet passage 103, and finally flows to the outlet port 110, to complete a smoking.

In summary, the cartridge, the atomizing component and the electronic cigarette of the embodiment have the following beneficial effects:

1. When smoking, the airflow enters from the outer periphery of the cartridge 1 and passes through the atomizing head 2, the airflow enters the bottom of the atomizing head 2 firstly, then exits from the top of the atomizing head 2, and finally exits through the top of the cartridge 1, to complete a smoking. The intake passage 102 and the smoke outlet passage 103 are disposed on the same side, the airflow enters at the upper end and flows out at the upper end, and are not leaked, so that the cartridge 1 and the atomizing head 2 are easily replaced. In addition, the lower end channel of the smoke outlet passage 103 is narrowed relative to the upper end passage, the smoke outlet passage 103 is generally trumpet-shaped with a larger, upper portion and smaller, lower portion, which is favorable for forming a negative pressure during smoking, and the smoking effect is better.

2. Two oppositely disposed Inserting members 13 are provided in the smoke outlet passage 103 to reduce the volume of the smoke outlet passage 103, the atomized smoke liquid flows from the gap between the two Inserting members 13 to the outlet port 110, thereby reducing exhaust gas flow and increasing the sensitivity of the sensor 6.

3. The rubber plug 5 corresponding to the inlet channel 102 is provided with a recess 50, the recess 50 is in communication with the inlet channel 102. A sensor 6 is mounted in the recess 50, the sensor 6 protrudes downward to form a connecting portion 60, and the connecting portion 60 protrudes from the bottom of the rubber plug 5. The rubber plug 5 can create a negative pressure sensing space for the sensor 6 inside the main body 3, and can also prevent the condensed smoke droplets from falling onto the main board 70 and damaging the main board 70.

4. The bottom side of the main body 10 is provided with a locking hole 109 on two lateral sides thereof. A buckle 30 is protruded from the two card holes on the two sides of the top of the main body 3, each of the buckles 30 is correspondingly inserted into one of the locking holes 109. By providing the buckle 30 on the main body 3, the size of the buckle 30 can be made larger, thereby effectively preventing the cartridge 1 from being detached from the electronic control assembly 7.

### The Second Embodiment

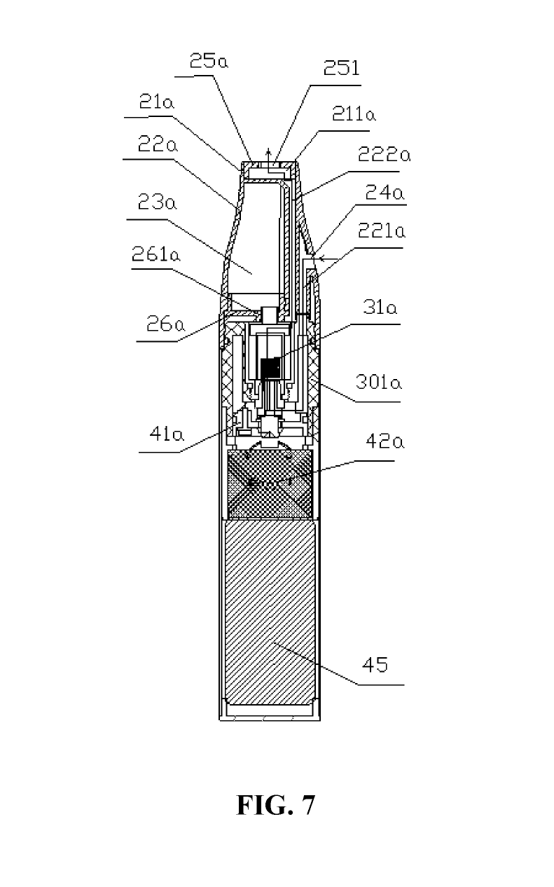

Referring to FIG. 6 and FIG. 7, the embodiment provides an electronic cigarette including an shell 10a, a cartridge 20a mounted on the top end of the shell 10a, and an atomizing assembly 30a and an electronic control assembly 40a installed in the shell 10a. The cartridge 20a supplies the smoke liquid to the atomizing assembly 30a, the atomizing assembly 30a atomizes the smoke liquid flowed therein. The electronic control assembly 40a is mounted on the bottom side of the atomizing assembly 30a to supply power to the atomizing assembly 30a and control the operation of the electronic cigarette.

The peripheral wall of the shell 10a is provided with a button hole 11a and a USB port 12a. A trigger button 13a is installed in the button portion 11a. The USB interface 12a is configured to charge the battery by an external power source.

The cartridge 20a has a bottle neck shape, and its bottom end gradually contracts toward the top end. The cartridge 20a includes a top wall 21a and a peripheral wall 22a, the top wall 21a and a peripheral wall 22a are correspondingly enclosing a reservoir chamber 23a. Further, cartridge housing (not shown) includes the top wall 21a and the peripheral wall 22a. The top end of the top wall 21a is provided with a receiving groove 211a, the receiving groove 211a is not in communication with the reservoir chamber 23a. The reservoir chamber 23a is closed at the top end, the reservoir chamber 23a has an opening at the bottom end thereof, the opening can be an integral through hole. The thickness of one side wall of the peripheral wall 22a is thicker than the other side wall, it can be considered that the peripheral wall 22a includes a thickness wall side and a thin wall side, the peripheral wall can be composed of the thickness wall side and the thin wall side. Or, the peripheral wall is provided with only one thickness wall portion (which can be regarded as the wall thickness side), the rest of the peripheral wall is formed by a thin wall portion (which can be regarded as a thin wall side). The thickness wall side can be the half circumferential side of the cartridge 20a. An air intake passage 221a and a smoke outlet passage 222a are formed on the thickness wall side in the axial direction of the cartridge 20a, that is, the intake passage 221a and the smoke outlet passage 222a are formed on the same side of the cartridge 20a. Further, the cartridge 20a is provided with a smoke outlet passage 222a and an intake passage 221a the same side thereof, the intake passage 221a and the smoke outlet passage 222a are disposed on the same side of the reservoir chamber 23a. Further, the reservoir chamber 23a, the smoke outlet passage 222a, and the intake passage 221a are sequentially arranged side by side. In one embodiment, a partition wall is formed between the intake passage 221a and the smoke outlet passage 222a. The intake passage 221a is adjacent to the outer peripheral surface of the cartridge 20a, the smoke outlet passage 222a is adjacent to the reservoir chamber 23a. An air inlet 24a is correspondingly defined on the peripheral wall 22a to communicate with the top end of the intake passage 221a, so that after being supplied from the air inlet 24a, airflow can flow out from the bottom end of the peripheral wall along the intake passage 221a. The smoke passage 222a extends axially from the bottom end of the peripheral wall 22a to the top end of the peripheral wall 22a, the top thereof is in communication with the receiving groove 211a, so that the airflow enters via the bottom end of the smoke outlet passage 222a and flows out from the top end of the peripheral wall 22a. The top of the smoke outlet passage 222a is narrowed relative to the receiving groove 211a. Further, the receiving groove 211a is provided with a smoke guiding cover 25a for connecting an external cigarette holder (not shown), the center of the smoke guiding cover 25a is provided with a smoke guiding hole 251a, the airflow flowing into the receiving groove 211a is flows out via the smoke guiding hole 251a, corresponding to the center position of the top end of the cartridge 20a. The bottom open end of the reservoir chamber 23a can be further provided with a baffle 26a, the liquid inlet 261a is defined in the baffle 26a for the smoke liquid in the reservoir chamber 23a to flow into the atomizing assembly 30a. The receiving groove 211a can function as a buffer to cool down and prevent hot to the mouth. It can be understood that the peripheral wall of the cartridge 20a can be integrally provided as a thick wall (regardless of the amount of material or the weight of the cartridge, etc.). That is, the intake passage 221a and the smoke outlet passage 222a are provided with a sufficient thickness, and it is only necessary to ensure that the intake passage 221a and the smoke outlet passage 222a are defined at the same side of the peripheral wall. By defining the air passage 221 and the smoke outlet passage 222a at the same side of the peripheral wall, the length of the air passage from the intake port to the outlet is shortened, thereby effectively solving the problem that the user needs to pump vigorously to suck out the smoke, the user inhale is more convenient and labor-saving.

The atomizing assembly 30a is mounted at the bottom side of the cartridge 20a, the two ends of the atomizing assembly 30aa are connected to the outlet of the intake passage 221a and the inlet of the smoke outlet passage 222a, respectively. The atomizing assembly 30a atomizes the smoke liquid flowing from the reservoir chamber of the cartridge 20a to generate smoke, so that the smoke can be carried out by the airflow. The atomizing assembly 30a includes a main body 301a and an atomizing head 31a installed in the main body 301a. The main body 301a has a tubular shape and is mounted in the shell 10a, the upper end of the main body 301a is tightly coupled to the cartridge 20a. Optionally, the liquid inlet 261a is disposed adjacent to the smoke outlet passage 222a, the atomizing head 31a is disposed below the liquid inlet 261a and is in communication with the liquid inlet 261a, thereby shortening the distance between the atomizing head 31a and the smoke outlet passage 222a, improving the atomization effect and increasing the amount of smoke.

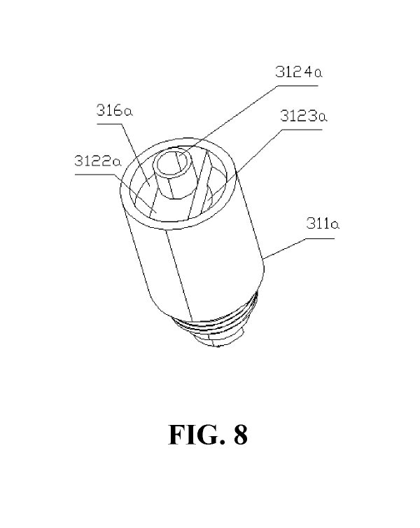

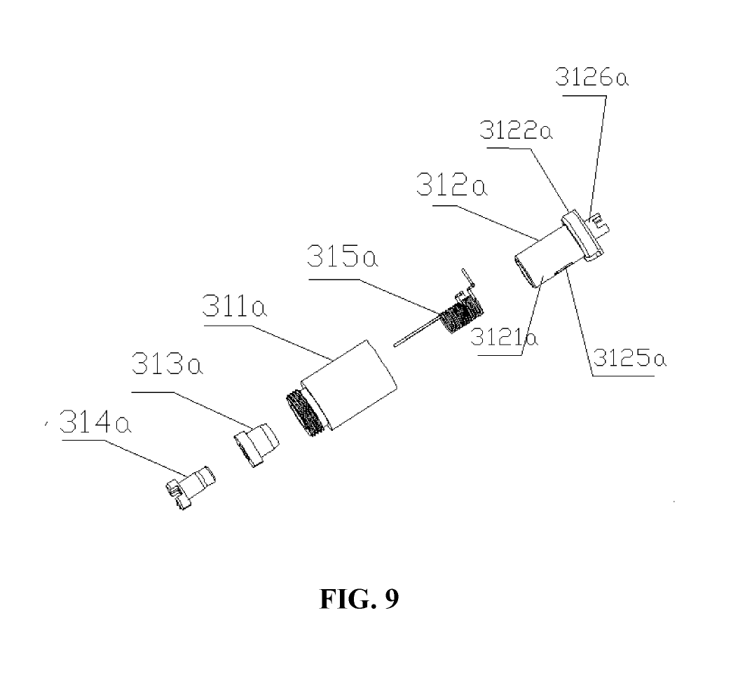

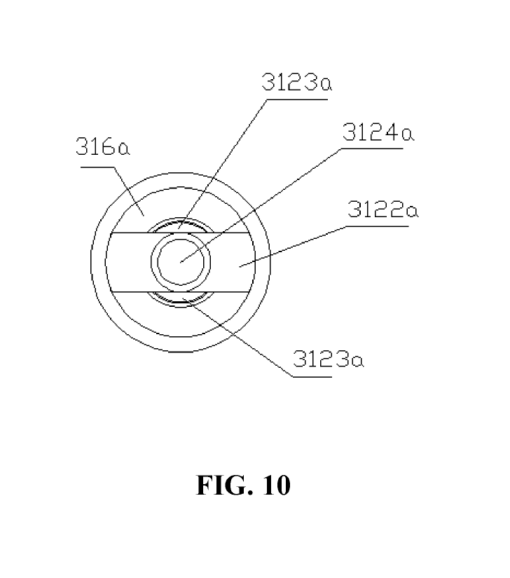

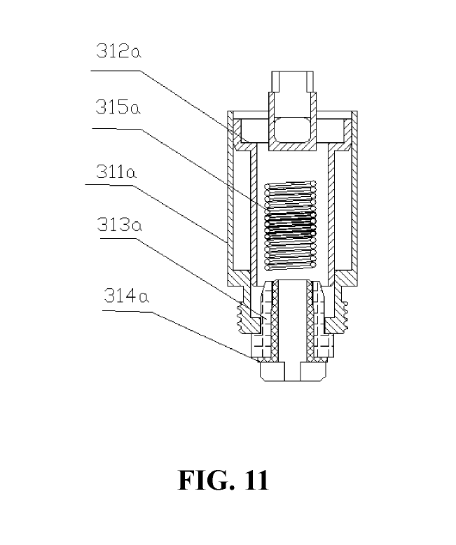

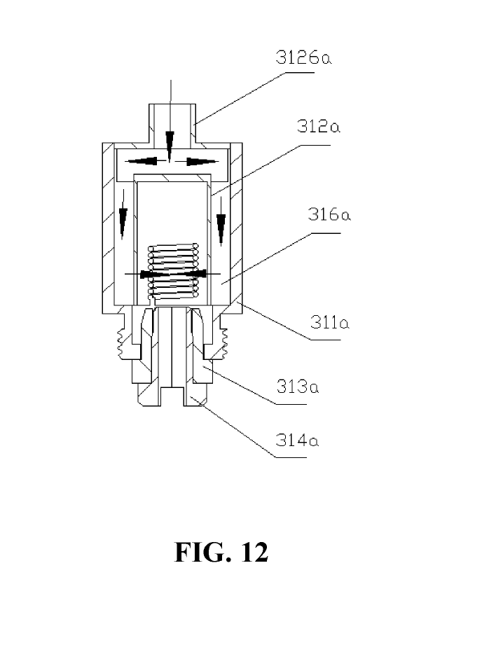

Referring to FIGS. 8 and 9, the atomizing head 31a includes a reservoir tube 311a, an inner sleeve 312a, an insulating member 313a, a conductor 314a and a heating element 315a. The reservoir tube 311a is for storing the e-cigarette liquide-cigarette liquid flowing in from the cartridge and serving as a conductive member at the same time, for example, the atomizing head is electrically connected to one end of the electrode, which has an opposite polarity relative to the end connected to the conductor 314a. One end of the reservoir tube 311a is formed as a threaded end for the reservoir tube 311a to be stably installed in the main body 301a. The inner sleeve 312a is mounted in a reservoir tube 311a, the inner sleeve 312a includes an atomizing tube 3121a and a liquid guiding tube 3122a radially disposed at one end of the atomizing tube 3121a. The pipe of the atomizing tube 3121a is an atomizing heating channel. The liquid guiding tube 3122a is disposed at the top end of the atomizing tube 3121a, both ends of the liquid guiding tube 3122a protrude from the peripheral wall side of the atomizing tube 3121a, the width of the liquid guiding tube 3122a is narrower than the diameter of the atomizing tube 3121a, thereby correspondingly, a smoke outlet 3123a is formed at the port of the atomizing tube 3121a and is located at two sides of the catheter 3122a. The length of the liquid guiding tube 3122a is equivalent to the inner diameter of the reservoir tube 311a, the inner sleeve 312a is installed in the reservoir tube 311a, both ends of the liquid guiding tube 3122a are connected to the inner circumferential surface of the reservoir tube 311a (for example, screwing connection), a liquid storage gap 316a is formed between the outer circumferential surface of the atomizing tube 3121a and the inner circumferential surface of the reservoir tube 311a (refer to FIG. 10 in combination). Referring to FIG. 11, the atomizing tube 3121a is folded outward at a position flush with the bottom of the liquid guiding tube. After the folding, the atomizing tube 3121a abuts against the liquid storage tube, thereby sealing the top of the liquid storage gap 316a. Referring to FIG. 12, a liquid drop hole (not shown) is respectively disposed on the bottom surfaces of the two ends of the liquid guiding tube 3122a extending from the atomizing tube 3121a to connect the liquid guiding tube 3122a tube with the liquid storage gap 316a. The top surface of the liquid guiding tube 3122a is provided with a Liquid injecting port 3124a for allowing the smoke liquid flow into the reservoir chamber 23a. In the present embodiment, the liquid pipe 3126a is provided with a liquid pipe 3126a at the top end of the liquid guiding pipe 3122a, the nozzle of the liquid pipe 3126a is the Liquid injecting port 3124a. Optionally, the liquid pipe 3126a has a circular cross section, the liquid pipe 3126a is in communication with the pipe of the liquid guiding pipe 3122a, the liquid pipe 3126a cooperates with the liquid inlet 261a defined at the bottom of the reservoir chamber 23a for allowing smoke liquid flow into the liquid guiding tube 3122a and flow into the liquid storage gap 316a. In addition, a liquid inlet hole 3125a is formed in the pipe wall of the atomizing pipe 3121a, so that the e-cigarette liquide-cigarette liquid in the liquid storage gap 316a can flow into the atomizing pipe 3121a to be atomized.

The insulating member 313a is sleeved on the conductor 314a and installed at one end of the reservoir tube 311a, so that the conductive member 314 is insulated from the reservoir tube 311a. The conductor 314a and the insulating member 313a are respectively provided with a shaft hole (not shown) for introducing airflow from the bottom into the atomizing tube 3121a. The heating element 315a is mounted in the atomizing tube 3121a, one end of the heating element 315a is connected to the reservoir tube 311a, and the opposite end of the heating element 315a is connected to the conductor 314a. In the reservoir tube 311a and the conductor 314a, one of them is connected to the positive pole and the other is connected to the negative pole. Further, the outer circumference of the heating element 315a can cover the liquid guiding element (not shown), the liquid guiding element is wrapped around the outer circumference of the heating element, and is integrally mounted in the inner cavity of the atomizing tube 3121a. The heating element 315a is selected as a spiral formed by a spiral of a heating wire, the liquid guiding element is selected to from a liquid absorbent cotton or a fiber rope. Further, a heating element may be wrapped around the outer circumference of the liquid guiding member.

After the atomizing head 31a is installed in the main body 301a, the atomizing head 31a is installed at the bottom end of the cartridge 20a, the top end of the reservoir tube 311a of the atomizing head 31a is sealed against the bottom surface of the partition wall between the air intake passage 221a and the air outlet passage 222a at the bottom of the cartridge 20a, the baffle 26a is sealed against the open end of the bottom of the reservoir chamber 23a, thereby, the intake passage 221a is isolated from the smoke outlet passage 222a. An air gap in communication with the intake passage 221a is formed between the outer circumferential surface of the reservoir tube 311a and the inner circumferential surface of the main body 301a. The air entering the intake passage 221a is introduced to the bottom of the atomizing head 31a through the air gap, and is introduced into the pipe of the atomizing tube 3121a from the axial hole of the conductive member 314. After the air passes through the pipe of the atomizing tube 3121a, the smoke is taken out from the smoke outlet 3123a, and flows out to the smoke outlet passage 222a.

Referring to FIG. 11 and FIG. 12, the smoke liquid flowing out of the reservoir chamber 23a flows through the liquid guiding tube 3122a into the liquid storage gap 316a, and enters the atomizing tube 3121a via the liquid inlet hole 3125a thereof, the inside of the tube 3121a is further atomized by the heating element 315a; the airflow of the intake passage 221a enters the atomizing heating passage, and the smoke in the atomizing heating passage is taken out through the smoke outlet passage 222a. The electronic control assembly 40a is responsive to inhaling and supplying power to the atomizing head 31a. The electronic control assembly 40a includes a mounting bracket 41a, a circuit board 42a and a power source 45a that are mounted in the shell 10a. The circuit board 42a is mounted on the mounting bracket 41a, the USB board is provided on the circuit board 42a to match the USB port 12a of the shell 10a for connection to the charger. A switch that matches the trigger button 13a is also disposed on the circuit board 42a to turn the heat generating component 315a on or off. The power source 45a is detachably mounted in the shell 10a below the circuit board 42a for supplying power to the circuit board 42a and the atomizing head 31a, the power source 45a may be a battery or a battery cell.

When installing the electronic cigarette atomizing head, the atomizing head 31a is first twisted into the main body 301a, and then the atomizing head 31a is inserted into the through hole at the bottom of the cartridge 20a; at the same time, the cartridge 20a and the main body 301a of the atomizing assembly are tightly buckled (snap-fitted). When smoking, press the trigger button 13a to smoke normally. When there is no power, it can be charged through the USB port 12a. The electronic cigarette cartridge of the embodiment is used as a disposable cigarette, the cartridge 20a does not have a liquid injection hole, the cartridge 20a cannot be refilled with smoke liquid. It can be understood that when it is required to be used multiple times, it is required to define a liquid injection hole (not shown) on the cartridge 20a, for example, a liquid injection hole is radially defined at the peripheral wall 22a, through which the smoke liquid is injected into the reservoir chamber 23a. The liquid injection hole is not connected to the air intake passage 221a or the smoke outlet passage 222a, so as to ensure that the smoke liquid can be directly injected into the reservoir chamber 23a, for example, liquid injection hole is defined at the peripheral wall 22a and does not in communication with the intake passage 221a or the smoke passage. 222a.

In summary, the airflow of the electronic cigarette provided in this embodiment enters from the outer periphery of the cartridge 20a and passes through the atomizing head 31a. The airflow first enters the bottom of the atomizing head 31a. Then venting from above the atomizing head 31a, finally, the gas is vented through the top end of the cartridge 20a, to complete a smoking. The Intake passage 221a and smoke outlet passage 222a are disposed at the same side, the airflow enters at the upper end and flows out at the upper end, and smoke liquid are not leaked, so that the cartridge 20a and the atomizing head 31a are easily replaced.

### The Third Embodiment

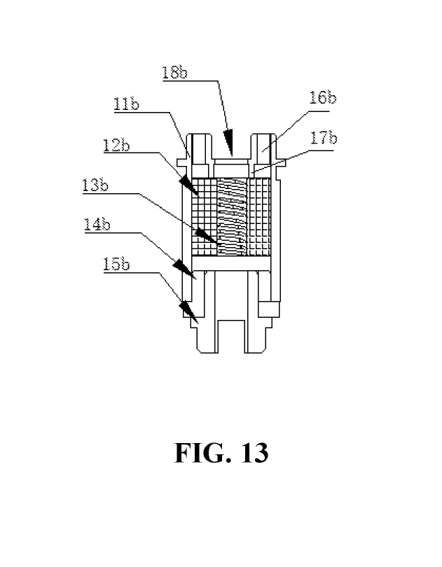

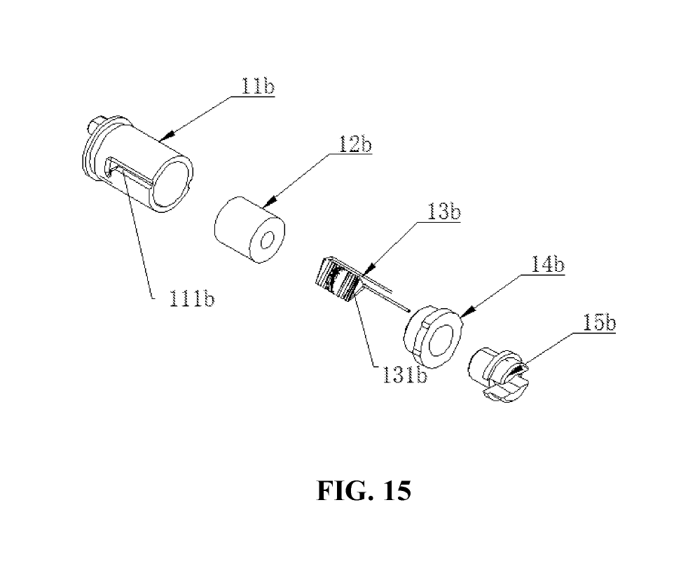

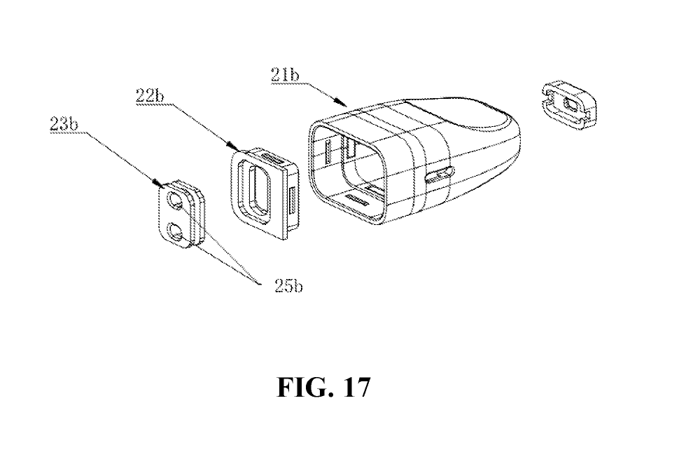

As shown in FIGS. 13-18, the embodiment discloses an electronic cigarette including a shell 40b, a battery assembly 30b and an atomizing assembly. The battery assembly 30b is disposed in the shell 40b for supplying power to the atomizing component. The atomizing assembly includes a cartridge 21b and an atomizer. The cartridge 21b is mounted on the upper end of the shell 40b. The atomizer is detachably fixed to the bottom end of the cartridge 21b and disposed within the shell 40b.

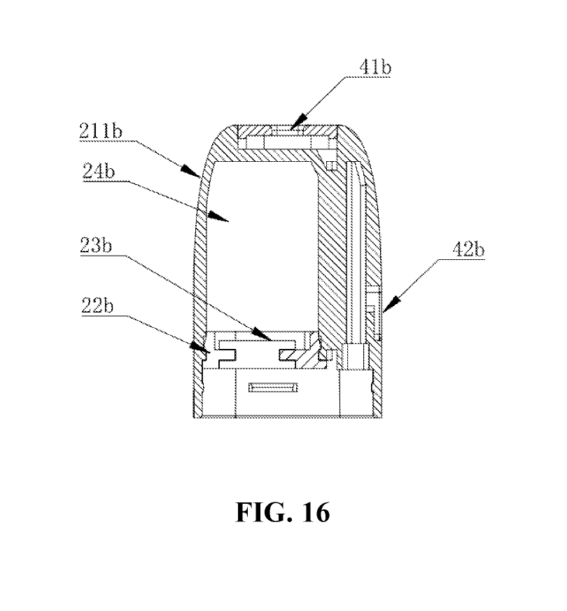

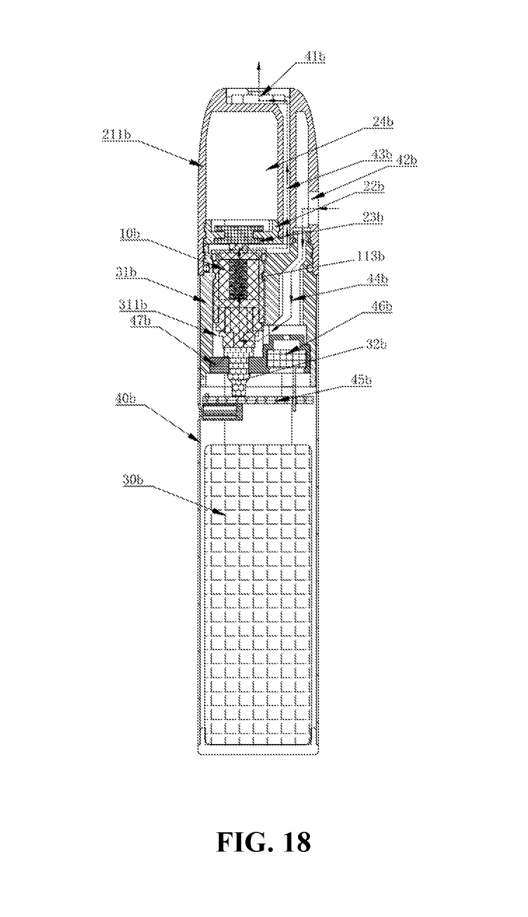

As shown in FIG. 16 and FIG. 18, the cartridge 21b includes a cartridge housing 211b, the cartridge housing 211b has a reservoir chamber 24b and a smoke outlet passage 43b. The bottom end of the reservoir chamber 24b is provided with a reservoir chamber hole 25b, the top end of the cartridge housing 211b is provided with a smoke outlet 41b, and one end of the smoke outlet passage 43b is in communication with the smoke outlet 41b.

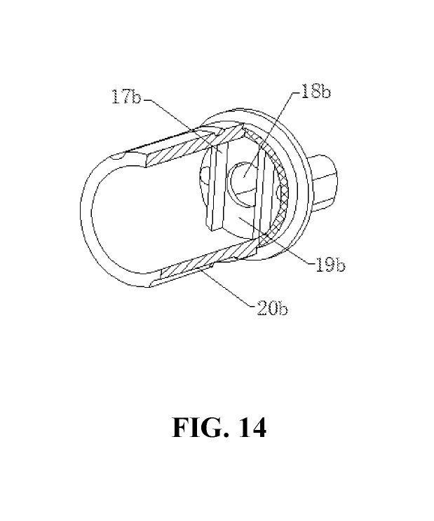

As shown in FIG. 18, the atomizer includes an atomizing head main body 31b and an atomizing head 10b. The atomizing head main body 31b is partially received at the bottom end of the cartridge housing 211b and fixedly connected to the cartridge housing 211b, the remaining of the atomizing head main body 31b is partially disposed in the shell 40b, the atomizing head main body 31b is provided with an atomizing head mounting hole 311b, the atomizing head 10b is disposed in the atomizing head mounting hole 311b. In one embodiment, the bottom of the reservoir chamber 24b is provided with a sealing member 23b, the reservoir chamber hole 25b is disposed on the sealing member 23b. Specifically, the sealing member 23b is a silicone gasket or a rubber gasket. As shown in FIGS. 1 and 2, the atomizing head 10b includes an atomizing head housing 11b, a heating element 13b, and a liquid guiding member 12b. The atomizing head housing 11b includes a housing body 20b and an atomizing head upper cover 19b fixedly connected to the upper end of the housing body 20b, a cavity is formed inside the housing body 20b, the heating element 13b and the liquid guiding member 12b are both located in the cavity, the liquid guiding member 12b is sleeved on the outer circumference of the heating element 13b. The atomizing head upper cover 19b is provided with a liquid inlet hole 16b in communication with the cavity, the liquid guiding member 12b is located at the lower end of the liquid inlet hole 16b to ensure that the smoke liquid flowing through the liquid inlet hole 16b can be introduced into the liquid guiding member 12b. A blocking portion 17b is disposed between the heating element 13b and the liquid inlet hole 16b. The upper end surface of the blocking portion 17b abuts against the lower end surface of the atomizing head upper cover 19b, the lower end surface of the blocking portion 17b abuts against the upper end surface of the liquid guiding member 12b. Alternatively, the blocking portion 17b is integrally formed with the atomizing head housing 11b. The liquid inlet hole 16b is in communication with the reservoir chamber hole 25b at the bottom end of the reservoir chamber 24b. It should be noted that the function of the blocking portion 17b is to block the direct flow of the e-cigarette liquid flowing from the liquid inlet hole 16b to the heating element 13b, so that the smoke liquid can only flow to the liquid guiding member 12b, and then is conducted to the surface of the heating element 13b through the liquid guiding member 12b. Therefore, the arrangement of the blocking portion 17b can be as shown in FIG. 14, that is, including two strip-shaped blocking portions 17b. It can be understood that when the liquid inlet hole 16b includes two or more (excluding two), the corresponding number of the blocking portions 17b can be correspondingly set according to the above setting manner. Alternatively, two or more (excluding two) of the above-mentioned liquid inlet holes 16b are respectively disposed on both sides of the two blocking portions 17b. In one embodiment, the blocking portion 17b is an annular body, and disposed around the top end of the heating element 13b, at this time, the liquid inlet hole 16b can be any number and are disposed outside of the blocking portion 17b. Alternatively, the blocking portion 17b may have a closed shape such as a quadrangle or a pentagon. Further, in one embodiment, The blocking portion 17b extends downward in the axial direction of the liquid inlet hole 16b, and the lower end is formed to abut against the liquid guiding member 12b, at this time, the liquid inlet hole 16b can be any number, the shape of the blocking portion 17b depends on the shape of the liquid inlet hole 16b, the blocking portion 17b is disposed around the liquid inlet hole 16b, as long as the smoke liquid flowing through the liquid inlet hole 16b flows through the liquid guiding member 12b and then contacts the heating element 13b. In one embodiment, the blocking portion 17b is a polygonal shape that acts as a sealing member.

As shown in FIGS. 13-15 and 17-18, an air outlet 18b is further disposed on the atomizing head upper cover 19b of the atomizing head, an atomizing head air passage 131b is formed inside the heating element 13b, the atomizing head air passage 131b is in communication with the outside air. Specifically, one end of the atomizing head air passage 131b is connected to the air outlet 18b, the air outlet 18b is in communication with the smoke outlet passage 43b and the smoke outlet hole 41b of the cartridge housing 211b.

As shown in FIG. 18, the cartridge 21b is also connected with the atomizing head main body 31b to form an air flow passage 44b. An air inlet 42b in communication with the air flow passage 44b is provided on an outer peripheral wall of the cartridge housing 211b, the smoke bomb 21b is formed on the same side with a smoke outlet passage 43b and an intake passage (not shown), the intake passage and the smoke outlet passage 43b are disposed on the same side of the reservoir chamber 24b.

Further, the reservoir chamber 24b, the smoke outlet passage 43b, and the intake passage are sequentially arranged side by side, the air inlet 42b is in communication with the intake passage, the other end of the atomizing head air passage 131b is in communication with the air flow passage 44b. A matching portion 113 is disposed in the atomizing head mounting hole 311b. The atomizing head 10b is correspondingly provided with a limiting portion 111, the atomizing head 10b is fixed in the atomizing head mounting hole 311b by the matching portion 113. Specifically, the limiting portion 111 is an L-shaped groove formed on the outer peripheral wall of the housing body 20b; corresponding to the position of the L-shaped groove, the inner peripheral wall of the atomizing head mounting hole 311b is provided with a protrusion that cooperates with the L-shaped groove, the protrusion is the matching portion 113. Alternatively, the limiting portion 111 is a protrusion formed on the outer peripheral wall of the housing body 20b; corresponding to the position of the protrusion, the inner peripheral wall of the atomizing head mounting hole 311b is provided with an L-shaped groove that cooperates with the protrusion, the L-shaped groove is the fitting portion 113. The atomizing head 10b in this embodiment is mounted and disassembled as follows: one end of the atomizing head 10b is opposite to the atomizing head mounting hole 311b, the protrusion is directly opposite to the entrance of the L-shaped groove. At this time, the atomizing head 10b can be pushed into the atomizing head mounting hole 311b, the projection travels along the L-shaped groove until the bottom end of the groove, which in turn can rotate the atomizing head 10b to a certain mounting angle. Therefore, the matching structure of the above-mentioned protrusion and the L-shaped groove has a guiding function and a positioning function.

As shown in FIG. 18, the electronic cigarette further includes a sensor 46b, a sealing gasket 47b is disposed between the battery assembly 30b and the atomizing assembly, a sealing gasket 47b is fixed to the bottom end of the atomizing head main body 31b. The sensor 46b is fixed in the sealing gasket 47b, the sensor 46b is in communication with the air flow passage 44b and is electrically connected to the battery assembly 30b.

Further, the electronic cigarette further includes a circuit board 45b, the circuit board 45b is disposed between the atomizer and the battery assembly 30b. In one embodiment, the sensor 46b, the atomizer and the battery assembly 30b are both electrically connected to the circuit board 45b. The sensor 46b, the atomizer, and the battery assembly 30b realize circuit conduction and signal transmission between two of the three through the circuit board 45b.

Optionally, the sensor 46b is a pressure sensing sensor, the air passage of the pressure sensing sensor is connected to the air flow passage 44b. Therefore, when the air flow passage 44b supplies air into the atomizer 11, the gas in the air passage of the pressure sensing sensor is taken away, causing a negative pressure, and the pressure sensing sensor generates an electric signal and transmits the electric signal to the circuit board 45b. The battery assembly. 30b includes a battery (not shown), the circuit board 45b controls the battery to supply power, and to make the electronic cigarette operate.

The electronic cigarette of the embodiment, the reservoir chamber 24b is configured for storing the smoke liquid, and then the smoke liquid flows into the atomizing head 10b via the reservoir chamber hole 25b and the liquid inlet hole 16b. In the atomizing head 10b, the liquid inlet hole 16b is directly aligned with the liquid guiding member 12b and is further provided with a blocking portion 17b. Therefore, the smoke liquid must be infiltrated from the liquid guiding member 12b to the heating element 13b, avoiding the technical problem that the heating element 13b is immersed in the smoke liquid. In addition, a sealing gasket 47b is provided between the middle battery assembly 30b and the atomizing assembly, the sealing gasket 47b is also located between the circuit board 45b and the atomizer, to block the condensate and the smoke liquid falling onto the circuit board 45b which can cause the circuit board 45b to be damaged.

The above-mentioned embodiments merely represent several implementations of the present application, and the descriptions thereof are more specific and detailed, but they shall not be understood as a limitation on the scope of the present application. It should be noted that, for those of ordinary skill in the art, variations and improvements may still be made without departing from the concept of the present application, and all of which shall fall into the protection scope of the present application. Therefore, the scope of protection of the present application shall be subject to the appended claims.

What is claimed is:

1. A cartridge, comprising:
a cartridge housing,
a reservoir chamber,
an intake passage, and
a smoke outlet passage, the reservoir chamber, the intake passage, and the smoke outlet passage are received in the cartridge housing, the intake passage and the outlet passage are disposed at the same side of the reservoir chamber.

2. The cartridge according to claim 1, wherein the reservoir chamber, the intake passage and the smoke outlet passage are sequentially arranged side by side.

3. The cartridge according to claim 1, wherein the cartridge comprises a top wall and a peripheral wall, the top wall and a peripheral wall are correspondingly enclosing a reservoir chamber, the peripheral wall of the cartridge is provided with a smoke outlet passage and an intake passage the same side.

4. The cartridge according to claim 2, wherein an air inlet is defined at the peripheral wall of the cartridge, the air inlet is in communication with the Intake passage.

5. The cartridge according to claim 1, wherein a partition wall is formed between the intake passage and the smoke outlet passage.

6. The cartridge according to claim 1, wherein the lower end passage of the smoke outlet passage is narrowed relative to the upper end passage.

7. The cartridge according to claim 1, wherein an inserting member is disposed in the smoke outlet passage, after the inserting member is inserted into the smoke outlet passage, the smoke outlet passage is substantially generally trumpet-shaped having a bigger, upper part and a smaller, lower part.

8. The cartridge according to claim 1, wherein the cartridge further comprising a main body, an upper cover and a bottom cover, the upper cover is provided with a smoke outlet, the smoke outlet passage is in communication with the smoke outlet.

9. The cartridge according to claim 1, wherein the inner wall of the outlet passage is provided with a slope or a bump, the outlet passage is provided with a slope or a projection, so that the outlet passage is substantially generally trumpet-shaped having a bigger, upper part and a smaller, lower part.

10. The cartridge according to claim 7 wherein the two inserting members have a cylindrical shape at the lower end and a wedge shape at the upper end, or the insert is a cylinder, the outer wall of the insert is axially attached to the inner wall of the outlet passage, the inserting member has a tapered hole penetrating vertically inside, the diameter of the tapered hole gradually decreases from top to bottom.

11. An atomizing assembly, comprising
a cartridge, comprises a cartridge housing, a reservoir chamber, a intake passage, and a smoke outlet passage, the reservoir chamber, the intake passage, and the smoke outlet passage are received in the cartridge housing, the intake passage and the outlet passage are disposed at the same side of the reservoir chamber;
an atomizer detachably secured to the bottom end of the cartridge.

12. An atomizing assembly according to claim 11, wherein the atomizing head comprises an atomizing head housing, a heating element, and a liquid guiding member, the atomizing head housing comprises a housing body and an atomizing head upper cover, a cavity is defined inside the housing body, the heating element and the liquid guiding member are both located in the cavity, the liquid guiding member is sleeved on the outer circumference of the heating element, the atomizing head upper cover is provided with a liquid inlet hole in communication with the cavity, the liquid guiding member is located at the lower end of the liquid inlet hole to ensure that the smoke liquid flowing through the liquid inlet hole can be introduced into the liquid guiding member, a blocking portion is disposed between the heating element and the liquid inlet hole, the upper end surface of the blocking portion abuts against the lower end surface of the atomizing head upper cover, the lower end surface of the blocking portion abuts against the upper end surface of the liquid guiding member to prevent the smoke liquid flowing through the liquid inlet hole from directly contacting the heating element without the liquid guiding member.

13. An atomizing assembly according to claim 12, wherein the atomizer comprises an atomizing head main body, the atomizing head main body is provided with an atomizing head mounting hole, the atomizing head is disposed in the atomizing head mounting hole.

14. An atomizing assembly according to claim 13, wherein the outer peripheral wall of the housing body defines a limiting portion.

15. The atomizing assembly according to claim 14, wherein the atomizing head mounting hole is provided with a matching portion that is fixedly coupled with the limiting portion, the atomizing head is fixed in the atomizing head mounting hole by the matching portion.

16. The atomizing assembly according to claim 15, wherein
the limiting portion is an L-shaped groove formed on the outer peripheral wall of the housing body;
corresponding to the position of the L-shaped groove, the inner peripheral wall of the atomizing head mounting hole is provided with a protrusion that cooperates with the L-shaped groove, the protrusion is the matching portion;
or, the limiting portion is a protrusion formed on the outer peripheral wall of the housing body;
corresponding to the position of the protrusion, the inner peripheral wall of the atomizing head mounting hole is provided with an L-shaped groove that cooperates with the protrusion, the L-shaped groove is the fitting portion.

17. An atomizing assembly, comprising
a cartridge, comprises a cartridge housing, a reservoir chamber, a intake passage, and a smoke outlet passage, the reservoir chamber, the intake passage, and the smoke outlet passage are received in the cartridge housing, the intake passage and the outlet passage are disposed at the same side of the reservoir chamber;
an atomizer provided with an atomizing heating channel, one end of the atomization heating passage is in communication with the intake passage, the other end is in communication with the smoke outlet passage.

18. The atomizing assembly of claim 17, wherein the atomizing head comprises a reservoir tube and an inner sleeve sleeved in the liquid storage tube, the inner sleeve comprises an atomizing tube, the pipe of the atomizing tube is an atomizing heating channel.

19. The atomizing assembly according to claim 18, wherein the atomizing head comprises a heating element and a liquid guiding element, the heating element is wrapped around the outer circumference of the liquid guiding member, or the liquid guiding member is wrapped around the outer circumference of the heating element.

20. An electronic cigarette, comprising:
an atomizing assembly comprising a cartridge, the cartridge comprises a cartridge housing, a reservoir chamber, an intake passage, and a smoke outlet passage, the reservoir chamber, the intake passage, and the smoke outlet passage are received in the cartridge housing, the intake passage and the smoke outlet passage are disposed at the same side of the reservoir chamber; and an atomizer detachably secured to the bottom end of the cartridge.
